(19) United States
(12) Patent Application Publication
(10) Pub. No.: US 2026/0066132 A1
(43) Pub. Date: Mar. 5, 2026

(54) ESTIMATION DEVICE, ESTIMATION METHOD, AND RECORDING MEDIUM

(71) Applicant: NEC Corporation, Tokyo (JP)

(72) Inventors: Kosuke NISHIHARA, Tokyo (JP); Keisuke SUZUKI, Tokyo (JP); Yuki KOSAKA, Tokyo (JP); Fumiyuki NIHEY, Tokyo (JP); Mana HASHIMOTO, Tokyo (JP); Kentaro NAKAHARA, Tokyo (JP)

(73) Assignee: NEC Corporation, Tokyo (JP)

(21) Appl. No.: 19/292,094

(22) Filed: Aug. 6, 2025

(30) Foreign Application Priority Data
Aug. 29, 2024 (JP) 2024-147670

(51) Int. Cl. G16H 50/30; G16H 10/60; G16H 50/70

(52) U.S. Cl. CPC G16H 50/30; G16H 10/60; G16H 50/70

(57) ABSTRACT

An estimation device includes a reception unit that receives an input of a data set for each stage, an estimation unit that estimates a state transition probability based on a transition from a first distribution that is a data distribution in a first stage to a second distribution that is a data distribution in a second stage based on a cost determined for each piece of data in the first distribution and required for a transition to a transition destination, and an output unit that outputs an estimated state transition probability. The estimation device can support decision making regarding future states by predicting future states through the estimation of state transitions.

This application is based upon and claims the benefit of priority from Japanese Patent Application No. 2024-147670, filed on Aug. 29, 2024 the disclosure of which is incorporated herein in its entirety by reference.

### TECHNICAL FIELD

The present disclosure relates to an estimation device and the like.

### BACKGROUND ART

In the field of healthcare and the like, there is a technology for performing future analysis of information regarding a person.

JP 2016-095684 A discloses that, in clusters for each age group as a result of clustering medical data, a transition probability in a direction in which an age group passes is calculated between the clusters based on similarity between the clusters, and each cluster for each age group is associated with a transition probability between the clusters to construct a prediction model in which a health state of a subject transitions for each age group.

### SUMMARY

An object of the present disclosure is to provide an estimation device and the like capable of outputting a state transition probability in consideration of a tendency of a change in data in data transition between distributions.

An estimation device according to an aspect of the present disclosure includes a reception unit that receives an input of a data set for each stage, an estimation unit that estimates a state transition probability based on a transition from a first distribution that is a data distribution in a first stage to a second distribution that is a data distribution in a second stage based on a cost determined for each piece of data in the first distribution and required for a transition to a transition destination, and an output unit that outputs an estimated state transition probability.

An estimation method according to an aspect of the present disclosure includes receiving an input of a data set for each stage, estimating a state transition probability based on a transition from a first distribution that is a data distribution in a first stage to a second distribution that is a data distribution in a second stage based on a cost determined for each piece of data in the first distribution and required for a transition to a transition destination, and outputting an estimated state transition probability.

A non-transitory recording medium according to an aspect of the present disclosure recording a program for casing a computer to execute a process of receiving an input of a data set for each stage, a process of estimating a state transition probability based on a transition from a first distribution that is a data distribution in a first stage to a second distribution that is a data distribution in a second stage based on a cost determined for each piece of data in the first distribution and required for a transition to a transition destination, and a process of outputting an estimated state transition probability.

### BRIEF DESCRIPTION OF THE DRAWINGS

Exemplary features and advantages of the present invention will become apparent from the following detailed description when taken with the accompanying drawings in which:

FIG. 1 is a first block diagram illustrating an example of a functional configuration of an estimation device of the present disclosure;

FIG. 2 is a first flowchart for explaining an example of the operation of the estimation device of the present disclosure;

FIG. 3 is a second block diagram illustrating an example of a functional configuration of the estimation device of the present disclosure;

FIG. 4 is a diagram illustrating an example of a probability density distribution of the present disclosure;

FIG. 5 is a diagram for explaining an image when a transition from one probability density distribution to another probability density distribution of the present disclosure is solved as an optimal transport problem;

FIG. 6 is a second flowchart for explaining an example of the operation of the estimation device of the present disclosure;

FIG. 7 is a third block diagram illustrating an example of a functional configuration of the estimation device of the present disclosure;

FIG. 8 is a fourth block diagram illustrating an example of a functional configuration of the estimation device of the present disclosure;

FIG. 9 is a third flowchart for explaining an example of the operation of the estimation device of the present disclosure;

FIG. 10 is a fifth block diagram illustrating an example of a functional configuration of the estimation device of the present disclosure;

FIG. 11 is a fourth flowchart for explaining an example of the operation of the estimation device of the present disclosure;

FIG. 12 is a fifth flowchart for explaining an example of the operation of the estimation device of the present disclosure; and

FIG. 13 is a block diagram illustrating an example of a hardware configuration of a computer device that implements the estimation device of the present disclosure.

### EXAMPLE EMBODIMENT

Hereinafter, example embodiments of the present disclosure will be described with reference to the drawings.

### First Example Embodiment

An outline of an estimation device according to a first example embodiment will be described.

The estimation device of the present disclosure calculates a state transition probability between data using the accumulated data. In the present disclosure, an example of target data is health data that is data regarding health of a person. The health data may be, for example, a value of an inspection item in a medical examination, or may be information regarding an exercise habit of a person. The health data is not limited to this example.

The health data is accumulated in advance, for example. At this time, the accumulated health data may not be temporal data that is a result of continuous observation of a specific person for a predetermined period. The accumulated health data may be data at a predetermined time point of each of the plurality of persons. For example, the results of the medical examinations for 10,000 people in the year of t may be accumulated as health data. In the present disclosure, an example in which the estimation device estimates the data transition based on the health data will be mainly described, but the target data is not limited to this example.

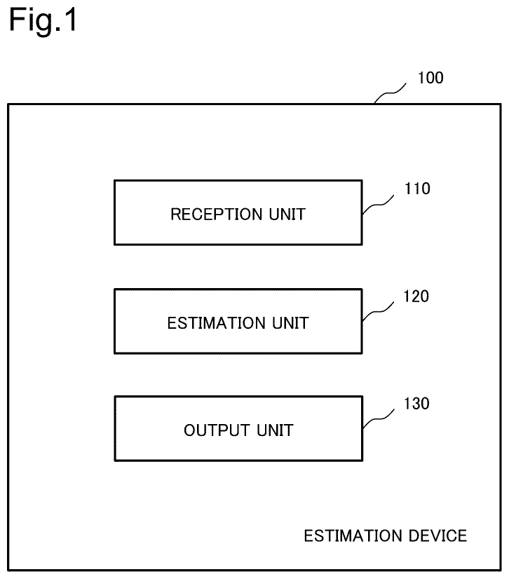

FIG. 1 is a first block diagram illustrating an example of a functional configuration of an estimation device 100. As illustrated in FIG. 1, the estimation device 100 includes a reception unit 110, an estimation unit 120, and an output unit 130.

The reception unit 110 receives an input of a data set. The data set may be health data of a plurality of persons. For example, the data set may be a result of a medical examination performed on a plurality of persons in one year. As described above, the data set may be data regarding the result of the medical examination at a predetermined time point of each of the plurality of persons. The data set may be stored in advance in a storage device (not illustrated). The storage device may be a device included in the estimation device 100 or an external device communicably connected to the estimation device 100.

The data sets are classified by stage. The stage may be information indicating a layer when the data set is stratified. In other words, the stage can also be said to be information indicating a condition in a case where data serving as a population is classified into a subset based on a predetermined condition. For example, if the data set is health data, the data set for each stage may be health data for each age group. More specifically, in a case where the health data is data indicating blood glucose levels of a plurality of persons, the data set for each stage may include data indicating a blood glucose level of a person whose age group is 10s, data indicating a blood glucose level of a person in 20s, . . . , and data indicating a blood glucose level of a person in 80s. That is, in this example, the data set includes data indicating blood glucose levels for every age group of 10s. In this manner, the order may be determined for the stage. For example, the next stage after the stage where the age group is 10s is the stage where the age group is 20s. The age group may be any age group.

In this manner, the reception unit 110 receives the input of the data set for each stage.

The estimation unit 120 estimates a state transition probability based on data transition between predetermined stages. Specifically, the estimation unit 120 estimates the state transition probability based on the transition from the data distribution in the first stage to the data distribution in the second stage. Here, the data distribution in the first stage is referred to as a first distribution. The data distribution in the second stage is referred to as a second distribution.

At this time, the estimation unit 120 estimates the state transition probability based on the cost required for the transition to the transition destination determined for each piece of data. For example, the estimation unit 120 estimates which data of the second distribution the data in the first distribution transitions to. At this time, a cost required for data transition is defined. The cost is determined according to the transition destination. For example, the estimation unit 120 estimates the state transition probability by obtaining a set of data in the first distribution and data in the second distribution so as to minimize the total cost required to transition each piece of data in the first distribution to the second distribution. At this time, the estimation unit 120 may estimate the state transition probability using an optimal transport algorithm.

The cost is determined for each piece of data. Therefore, for example, for each piece of data, the cost may be different even if the transition direction and the distance are the same. For the predetermined data, even if the transition distance is the same, the cost may be different depending on the transition direction. In other words, the cost may be asymmetric with respect to rising or falling values of the data. As a result, it is possible to determine the cost in consideration of a case where the ease of transition to the transition destination varies depending on the type and value of data.

It is assumed that the health data is data indicating blood glucose levels of a plurality of persons. It is assumed that the first stage is an age group of 40s and the second stage is an age group of 50s. That is, the second stage is a stage after the first stage. In this case, for example, the estimation unit 120 estimates the transition from the distribution of the blood glucose level of a person in 40s to the distribution of the blood glucose level of a person in 50s. Here, it is assumed that the blood glucose level is data that tends to deteriorate easily and hardly improve with aging. In this case, with respect to the data of the blood glucose level in the distribution in 40s, a cost that is likely to transition in the direction of deterioration and is difficult to transition in the direction of improvement is determined. In other words, regarding the blood glucose level data, a low cost is determined for the transition in the direction of deterioration, and a high cost is determined for the transition in the direction of improvement.

For example, using the cost information, the estimation unit 120 estimates a likely transition from the probability distribution indicating the probability that a person in 40s having each value of the blood glucose level is present to the probability distribution indicating the probability that a person in 50s having each value of the blood glucose level is present. In the estimation, a mapping from the probability distribution in 40s to the probability distribution in 50s is estimated. Estimating the mapping is synonymous with estimating a state transition probability related to a transition from a distribution of blood glucose levels of a person in 40s to a distribution of blood glucose levels of a person in 50s.

In this manner, the estimation unit 120 estimates the state transition probability based on the transition from the first distribution, which is the data distribution in the first stage, to the second distribution, which is the data distribution in the second stage, based on the cost required for the transition to the transition destination determined for each piece of data in the first distribution.

The output unit 130 outputs the estimated state transition probability. For example, the output unit 130 may output the state transition probability to an output device having a display or the like. For example, the output unit 130 may store the state transition probability in a storage device (not illustrated).

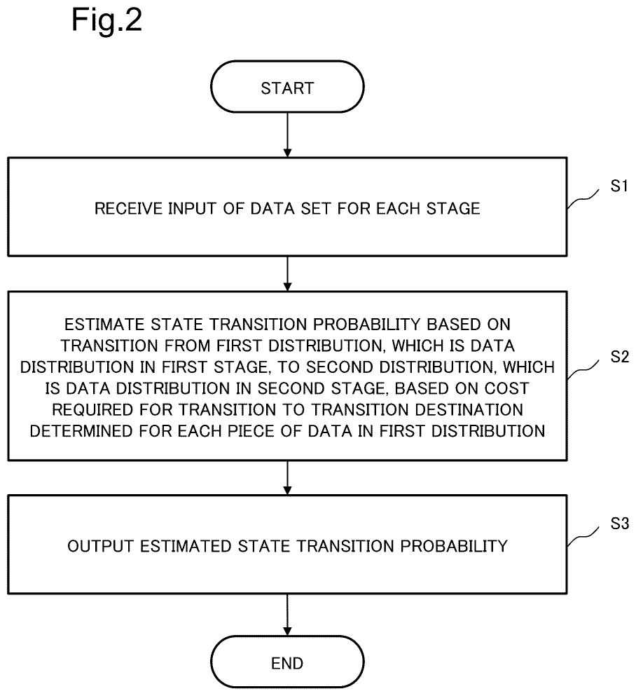

Next, an exemplary operation of the estimation device 100 will be described with reference to FIG. 2. In the present disclosure, each step of the flowchart is represented using a number assigned to each step, such as “S1”.

FIG. 2 is a flowchart for explaining an exemplary operation of the estimation device 100.

The reception unit 110 receives an input of a data set for each stage (S1).

The estimation unit 120 estimates the state transition probability based on the transition from the first distribution, which is the data distribution in the first stage, to the second distribution, which is the data distribution in the second stage, based on the cost required for the transition to the transition destination determined for each piece of data in the first distribution (S2).

The output unit 130 outputs the estimated state transition probability (S3).

As described above, the estimation device 100 of the first example embodiment receives the input of the data set for each stage. The estimation device 100 estimates the state transition probability based on the transition from the first distribution, which is the data distribution in the first stage, to the second distribution, which is the data distribution in the second stage, based on the cost required for the transition to the transition destination determined for each piece of data in the first distribution. Then, the estimation device 100 outputs the estimated state transition probability.

For example, for each piece of data in the first distribution, a cost relevant to the ease of transition to the transition destination or the like is set, so that the estimation device 100 can estimate the state transition probability based on the transition from the first distribution to the second distribution according to the tendency of the change in data. That is, the estimation device 100 can output the state transition probability in consideration of the tendency of the change in the data in the data transition between the distributions.

Therefore, the estimation device can support decision making regarding future states by predicting future states through the estimation of state transitions.

### Second Example Embodiment

Next, an estimation device according to a second example embodiment will be described. In the second example embodiment, a further example of the estimation device described in the first example embodiment will be described. Also in the second example embodiment, an example in which the estimation device 100 estimates the data transition based on the health data will be mainly described, but the target data is not limited to this example. Part of the description of content overlapping with that of the first example embodiment will be omitted.

### <Details of Estimation Device 100>

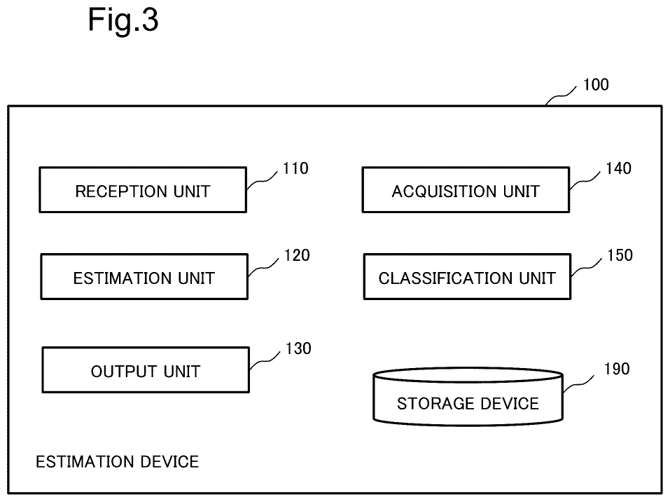

FIG. 3 is a block diagram illustrating an exemplary functional configuration of an estimation device 100. The estimation device 100 includes a reception unit 110, an estimation unit 120, and an output unit 130. The estimation device 100 may include an acquisition unit 140 and a classification unit 150. Further, the estimation device 100 may include a storage device 190. The storage device 190 may be a device included in the estimation device 100 or an external device communicably connected to the estimation device 100.

The estimation device 100 is, for example, a device provided in a terminal device such as a personal computer. The terminal device is a device operated by a user. The estimation device 100 is not limited to this example, and may be a device implemented in a server device communicably connected to a terminal device via a wired or wireless network. The estimation device 100 may perform various types of processing in accordance with an instruction from the terminal device.

The estimation device 100 may be communicably connected to a further device via a wired or wireless network. For example, the estimation device 100 may be communicable with an external server device having health data. The external server device is, for example, a device managed by a hospital, a local government, a company, or the like.

The reception unit 110 receives an input of a data set for each stage. At this time, the data set is stored in the storage device 190. For example, the reception unit 110 may receive reading of a data set stored in the storage device 190 as an input of the data set according to an instruction from the terminal device.

The storage device 190 stores health data. The reception unit 110 may receive the health data as a data set. The health data is acquired by the acquisition unit 140.

The acquisition unit 140 acquires health data. Specifically, the acquisition unit 140 acquires the health data from an external server device that manages the health data. For example, it is assumed that the results of the medical examination for 10,000 people in the year of t are managed by an external server device. The acquisition unit 140 acquires the results of the medical examination for 10,000 people in the year of t as health data from an external server device. The health data may be information relevant to an inspection item of a medical examination. The health data is a result of a medical examination that each of the 10,000 persons received at one time point in the year of t. As described above, the data set may be data measured at a time point of each of a plurality of targets instead of data indicating a temporal change of the same target. The acquisition unit 140 stores the acquired health data in the storage device 190.

The health data acquisition method is not limited to this example. For example, there may be a recording medium storing health data. At this time, the terminal device reads the health data from the recording medium. Then, the acquisition unit 140 may acquire the health data read by the terminal device.

In this manner, the acquisition unit 140 acquires health data that is data regarding health of each of a plurality of persons at a predetermined time point.

The health data is processed by the classification unit 150, for example. Then, the processed health data may be stored in the storage device 190. The classification unit 150 processes the data acquired by the acquisition unit 140 into a data set according to the condition. For example, the classification unit 150 classifies the health data for each age group. For example, the classification unit 150 classifies health data for each age group of 10s. The present invention is not limited to this example, and the classification unit 150 may classify the health data at an arbitrary age interval. For example, the classification unit 150 may classify the health data for every 1 year old.

At this time, the classification unit 150 may extract specific data from the health data and classify the extracted data for each age group. For example, it is assumed that the health data includes information indicating height, weight, blood pressure, blood glucose level, HbA1c, and Body Math Index (BMI). At this time, the classification unit 150 may classify data indicating the blood glucose level and BMI among the health data for each age group.

The condition to be classified and the data to be extracted may be information according to an instruction from the terminal device. That is, a user who operates the terminal device inputs information indicating a condition to be classified and data to be extracted to the terminal device. The terminal device transmits the input information to the estimation device 100. The classification unit 150 processes the health data using the information indicating the classification condition and the data to be extracted transmitted from the terminal device.

The classification unit 150 generates a distribution related to the acquired data. Specifically, the classification unit 150 generates a probability density distribution for each condition based on the acquired data. For example, the classification unit 150 generates a distribution obtained by plotting data indicating the blood glucose level and BMI for each age group. The distribution generated at this time is a two-dimensional distribution relating to blood glucose level and BMI. Then, the classification unit 150 generates a probability density distribution indicating an existence probability of each value of the blood glucose level and the BMI. When a data value of one dimension is xᵢ and a data value of another dimension is xⱼ, the probability density distribution can be expressed as p([xᵢ, xⱼ]). In this manner, the classification unit 150 generates a probability density distribution for each age group regarding the acquired health data.

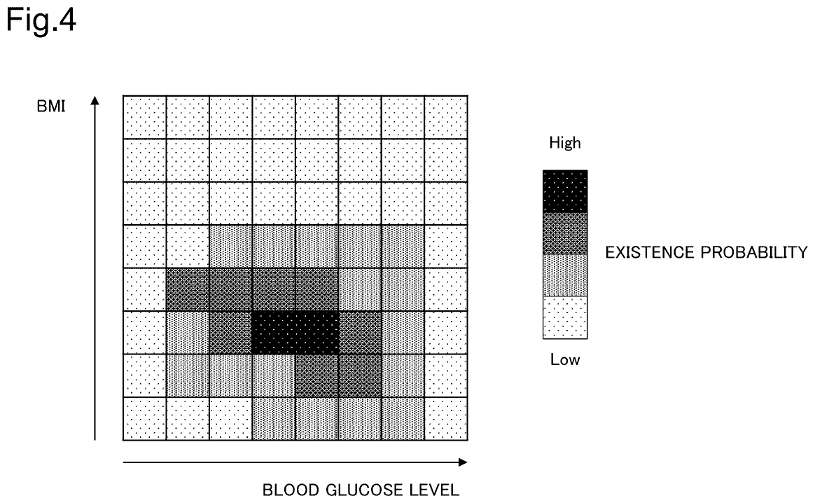

At this time, the classification unit 150 may classify data in each distribution into a data group. In this case, the probability density distribution is a distribution indicating the existence probability for each data group. FIG. 4 is a diagram illustrating an example of a probability density distribution. The probability density distribution illustrated in FIG. 4 is based on each of the blood glucose level and BMI. In the example of FIG. 4, 64 cells are illustrated. This cell is a data group in which data indicating the blood glucose level and BMI of each person is classified. Then, the existence probability for each data group is illustrated. In this manner, the classification unit 150 may classify data in each distribution of health data for each age group into a data group. The classification unit 150 may generate a two-dimensional or more distribution. For example, the classification unit 150 may generate a probability density distribution around each of the blood glucose level, BMI, and the average number of steps per day. The classification unit 150 may generate a probability density distribution regarded as continuously changing by reducing the width of the cell.

The reception unit 110 receives the health data classified for each condition as a data set for each stage. For example, the reception unit 110 may receive the probability density distribution related to the health data for each age group as described above as a data set for each stage.

The estimation unit 120 estimates the state transition probability between the stages. Specifically, the estimation unit 120 estimates the state transition probability based on the transition from the distribution of health data in the first age group to the distribution of health data in the second age group. Here, the second age group is an age group after the first age group. The distribution of the health data in the first age group is relevant to the first distribution. The distribution of the health data in the second age group is relevant to the second distribution.

The estimation unit 120 estimates the transition from the first distribution to the second distribution using an algorithm of an optimal transport problem (hereinafter, referred to as an optimal transport algorithm). The optimal transport algorithm is an algorithm for obtaining a transport method that optimizes a cost necessary for transitioning a predetermined probability distribution to another probability distribution.

Specifically, with respect to distributions μ and v in a probability space X, the fact that a distribution π in a direct product X² is coupling means that the following Expressions 1 and 2 hold.

$$π ( \cdot \times X ) = μ \qquad [ Math . 1 ]$$

$$π ( X \times \cdot ) = v \qquad [ Math . 2 ]$$

The entire coupling is defined as Π(μ, v). A cost function for transporting an element x included in the distribution u to an element y included in the distribution v is c(x, y). In this case, for example, in the following Expression 3, the coupling that minimizes the cost is referred to as optimal transport.

$${\int }_{{X}^{2}} c ( x , y ) π ( dxdy ) , π ∈ Π ( μ , v ) \qquad [ Math . 3 ]$$

Alternatively, assuming that the mapping from the distribution u to v is T(x), the direct transition may be obtained by finding T that minimizes the following Expression 4. In this case, Tis a one-to-one mapping. It is assumed that the volumes of the mapping T(U) and U are the same for the subset U of μ.

$${\int }_{{X}^{2}} c ( x , T ( x ) ) d μ ( x ) \qquad [ Math . 4 ]$$

When optimal transport is performed for discrete data, it can also be formulated as follows. Specifically, Cᵢⱼ is a cost matrix, and distributions are μᵢ and vⱼ. At this time, Pᵢⱼ that minimizes Expression 5 indicating the total cost is obtained under the condition shown in Expression 6.

$${{\sum }_{i = 1}}^{n} {{\sum }_{j = 1}}^{n} {C}_{ij} {P}_{ij} \qquad [ Math . 5 ]$$

$${{\sum }_{j = 1}}^{n} {P}_{ij} = {μ}_{i} , {{\sum }_{i = 1}}^{n} {P}_{ij} = {v}_{j} , {P}_{ij} \geq 0 , ∀ i , j \qquad [ Math . 6 ]$$

In this manner, it is possible to calculate a set of data before transport and data of a transport destination, which optimizes the cost of transport from the data distribution in the first stage to the data distribution in the second stage, by the optimal transport algorithm.

The estimation unit 120 estimates a state transition probability based on the transition from the first distribution to the second distribution. At this time, the estimation unit 120 solves the transition from the first distribution to the second distribution as the optimal transport problem. Here, the first distribution and the second distribution are probability density distributions. That is, the estimation unit 120 solves the transition from the probability density distribution related to the health data in the first age group to the probability density distribution related to the health data in the second age group as the optimal transport problem.

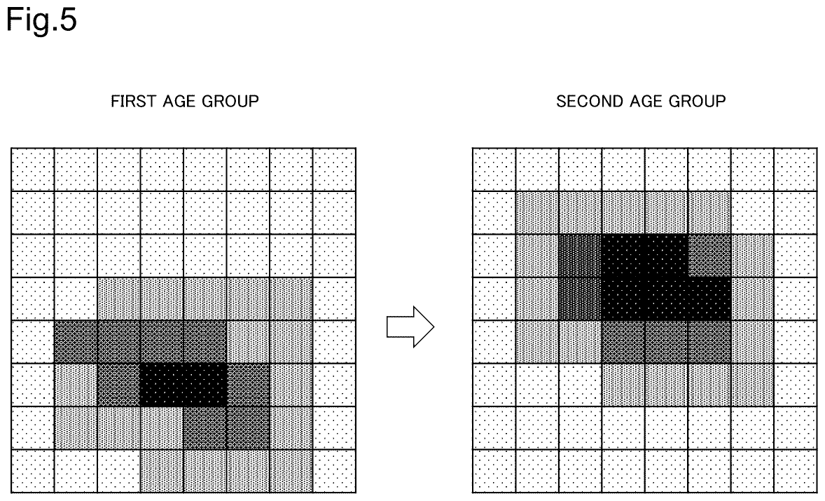

FIG. 5 is a diagram for explaining an image when a transition from one probability density distribution to another probability density distribution is solved as an optimal transport problem. FIG. 5 illustrates a probability density distribution related to health data in the first age group and a probability density distribution related to health data in the second age group. Solving, by the estimation unit 120, the transition from the first distribution to the second distribution as the optimal transport problem is relevant to estimating to which cell in the probability density distribution of the second age group the probability of transition of each cell in the probability density distribution of the first age group is higher. That is, the estimation unit 120 estimates the state transition probability based on the transition from each data group in the first distribution to each data group in the second distribution.

For example, u is a probability density distribution related to health data in the first age group, and v is a probability density distribution related to health data in the second age group. At this time, the estimation unit 120 estimates the mapping T using, for example, Expression 4. For example, the estimation unit 120 models the function of the mapping T with a neural network such as a fully-connected multilayer. The estimation unit 120 obtains the mapping T by performing optimization using machine learning so that Expression 3 becomes small. Furthermore, the estimation unit 120 generates a plurality of y transitioning from the given x using the mapping T. Then, the estimation unit 120 obtains the state transition probability from the generated y. Thus, the estimation unit 120 estimates the state transition probability.

The output unit 130 outputs the estimated state transition probability. For example, the output unit 130 may output the estimated state transition probability to the storage device 190 and store the state transition probability in the storage device 190.

Next, a specific example of the cost will be described. In equations such as Expressions 3 to 5, an equation related to cost such as a cost function or a cost matrix is determined. The cost may be determined according to a tendency of a change in data to be handled. Hereinafter, the cost function and the cost matrix may be collectively referred to as a cost function.

### [First Example of Cost]

In the first example, an example will be described in which the data to be handled has a specific change tendency in the data transition from the first stage to the second stage. Specifically, it is assumed that the health data is a test value in a medical examination or the like. For example, the health data is data regarding a blood glucose level, HbA1c, Mini Mental State Examination (MMSE), and the like. In this case, the health data tends to deteriorate easily and hardly improve with aging. Therefore, in the transition from the distribution of health data in the first age group (first distribution) to the distribution of health data in the second age group (second distribution), the cost required for the transition in the direction in which the health data deteriorates is set small, and the cost required for the transition in the direction in which the health data improves is set large.

In Expression 3, μ is a probability density distribution related to health data in the first age group, and v is a probability density distribution related to health data in the second age group. Health data in the distribution u is x, and health data in the distribution vis y. For example, it is assumed that the health data is data regarding HbA1c. At this time, a change in which the value of HbA1c increases is relevant to a transition in a direction in which health data deteriorates. A change in which the value of HbA1c decreases is relevant to a transition in a direction in which health data is improved. At this time, the cost function can be expressed by, for example, Expression 7 or Expression 8.

$$c ( x , y ) = exp \{ {\vert x - y \vert}^{2} - g ( x ) max ( y - x , 0 ) \} \qquad [ Math . 7 ]$$

$$c ( x , y ) = \frac{{\vert x - y \vert}^{2}}{1 + g ( x ) max ( y - x , 0 )} \qquad [ Math . 8 ]$$

In the expressions of Expression 7 and Expression 8, the value of the function g(x) is reflected with respect to the square error regarding the distance between the elements x and y. Here, g(x) in Expression 7 and Expression 8 may be different functions. g(x) can set an arbitrary function. In this example, a cost function is illustrated in which the cost is lowered in a case where the health data x in the first age group that is the stage before transition is increased to transition to the health data y in the second age group that is the stage after transition. That is, the cost function can be applied in a case where a change in which the health data rises is relevant to a transition in a direction in which the health data deteriorates. In a case where g(x) is set to a monotonically increasing function, it is expressed that the cost in a case where the health data x transitions to the health data y decreases as the value of the health data x increases. Similarly, by changing the role of g(x), it is possible to define a cost function that can be applied when a change in which the health data rises is relevant to a transition in a direction in which the health data improves.

In a case where the optimal transport is performed for discrete data as in Expression 6, the cost matrix can be expressed by the following Expression 9.

$${C}_{ij} = [ \begin{matrix}0 & 1 & 2 & 3 & 4 & 5 & 6 \\ 1 & 0 & 1 & 2 & 3 & 4 & 5 \\ 2 & 1 & 0 & 1 & 2 & 3 & 4 \\ 3 & 2 & 1 & 0 & 1 & 2 & 3 \\ 4 & 3 & 2 & 1 & 0 & 1 & 2 \\ 5 & 4 & 3 & 2 & 1 & 0 & 1 \\ 6 & 5 & 4 & 3 & 2 & 1 & 0\end{matrix} ] - g [ \begin{matrix}0 & 1 & 2 & 3 & 4 & 5 & 6 \\ 0 & 0 & 1 & 2 & 3 & 4 & 5 \\ 0 & 0 & 0 & 1 & 2 & 3 & 4 \\ 0 & 0 & 0 & 0 & 1 & 2 & 3 \\ 0 & 0 & 0 & 0 & 0 & 1 & 2 \\ 0 & 0 & 0 & 0 & 0 & 0 & 1 \\ 0 & 0 & 0 & 0 & 0 & 0 & 0\end{matrix} ] ( 0 < g < 1 ) \qquad [ Math . 9 ]$$

At this time, each of the first distribution and the second distribution may indicate a histogram indicating data included among data of seven types of values.

As described above, in the first example, it can be said that the cost function has asymmetry with respect to rising and falling of the value. The above-described cost function is merely an example. That is, the cost function is not limited to the above example.

As described above, in the transition of the health data from the first distribution to the second distribution, the estimation unit 120 may estimate the state transition probability based on the transition from the first distribution to the second distribution based on the cost function in which the cost of transition in the direction in which the health data of the first distribution deteriorates is smaller than the cost of transition in the direction in which the health data of the first distribution improves.

### [Second Example of Cost]

In the second example, an example in which a value relevant to the other axis has a specific change tendency according to a value relevant to a predetermined axis in the data transition between multidimensional distributions having at least two axes related to data will be described. For example, it is assumed that the health data is HbA1c and the average number of steps per day (hereinafter, simply referred to as “average number of steps”). That is, it is assumed that the first distribution and the second distribution are probability density distributions with HbA1c and the average number of steps as axes.

Here, it is assumed that HbA1c tends to be less likely to deteriorate and become better as the value of the average number of steps is larger. In this case, in the transition from the first distribution to the second distribution, a cost function in which the cost required for the transition is different according to the value of the average number of steps is set.

In Expression 3, u is a probability density distribution related to health data in the first age group, and v is a probability density distribution related to health data in the second age group. Health data in the distribution μ is x=(x₁, x₂), and health data in the distribution vis y=(y₁, y₂). At this time, it is assumed that the first axis is HbA1c and the second axis is the average number of steps. That is, x₁ and y₁ are values of HbA1c, and x₂ and y₂ are values of the average number of steps. In this case, for example, in the health data in which the value of HbA1c is a and the average number of steps is 5000 steps and the health data in which the value of HbA1c is a and the average number of steps is 10,000 steps, the cost required for the transition in the direction in which the value of HbA1c increases (deteriorates) is larger in the latter health data. That is, the latter health data is less likely to increase the value of HbA1c. At this time, the cost function can be expressed by, for example, Expression 10, Expression 11, or Expression 12.

$$c ( x , y ) = {d}^{2} + h ( {x}_{2} ) \qquad [ Math . 10 ]$$

$$c ( x , y ) = {d}^{2} \cdot h ( {x}_{2} + {y}_{2} ) \qquad [ Math . 11 ]$$

$$c ( x , y ) = \frac{{d}^{2}}{1 - h ( {x}_{2} - {y}_{2} )} \qquad [ Math . 12 ]$$

Here, d² is expressed by the following Expression 13.

$${d}^{2} = {\vert x - y \vert}^{2} = {\vert {x}_{1} - {y}_{1} \vert}^{2} + {\vert {x}_{2} - {y}_{2} \vert}^{2} \qquad [ Math . 13 ]$$

In Expressions 10 to 12, the value of the function h is reflected for the square error related to the distance between the elements x and y. Here, the functions h in Expressions 10 to 12 may be different functions. In this example, by setting h(x₂) to a monotonically increasing function of h(x₂)>0, it is expressed that the cost required for the transition in a direction in which the value of x₁ increases increases as the value of x₂ increases. That is, in the health data of the first distribution, it is expressed that the larger the value of the average number of steps, the more difficult it is to transition to a direction in which the value of HbA1c increases (a direction in which the value deteriorates). Similarly, by changing the role of the function h, it can be expressed that the smaller the value relevant to a specific axis, the more difficult it is to transition in a direction in which the value relevant to another axis increases.

As described above, in the second example, it can be said that the cost function has asymmetry with respect to a specific axis. By using a cost function asymmetric with respect to a specific axis, it is possible to estimate a state transition depending on a value of the specific axis. The above-described cost function is merely an example. That is, the cost function is not limited to the above example. The first distribution and the second distribution may be three-dimensional or more distributions having three or more axes.

As described above, it is assumed that the health data includes values of a plurality of health-related items of each of a plurality of persons. It is assumed that the first distribution and the second distribution are multidimensional distributions having each of a plurality of health-related items as an axis. In such a case, in the transition of the health data from the first distribution to the second distribution, the estimation unit 120 may estimate the state transition probability based on the transition from the first distribution to the second distribution based on the cost function in which the cost at which the health data of the first distribution transitions differs according to the magnitude of the value relevant to the specific axis.

### <Operation Example of Estimation Device 100>

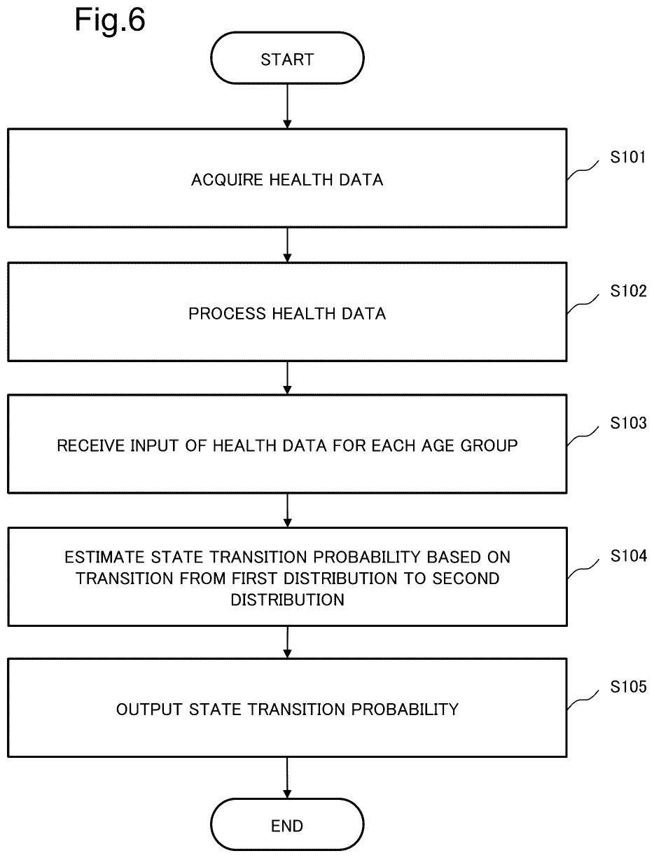

Next, an exemplary operation of the estimation device 100 will be described with reference to FIG. 6.

FIG. 6 is a second flowchart for explaining an exemplary operation of the estimation device 100. Specifically, FIG. 6 is a flowchart for explaining an exemplary operation when the estimation device 100 calculates the state transition probability between predetermined stages. In the present operation example, it is assumed that data indicating the blood glucose level and BMI is acquired as health data. At this time, it is assumed that the blood glucose level tends to be more deteriorated and less likely to be improved as the value of BMI is larger. The present operation example shows an example in which the estimation device 100 calculates a state transition probability from the distribution of health data in the age group of 40s to the distribution of health data in the age group of 50s.

The acquisition unit 140 acquires health data. For example, the acquisition unit 140 acquires health data from an external server device (S101). Then, the acquisition unit 140 stores the health data in the storage device 190. The classification unit 150 processes the health data (S102). For example, the classification unit 150 classifies the health data for each age group. Then, the classification unit 150 generates a probability density distribution for each age group of 10s regarding the health data. At this time, the probability density distribution is a distribution indicating the existence probability of each value of the blood glucose level and BMI. That is, the probability density distribution is a distribution around each of the blood glucose level and BMI.

The reception unit 110 receives an input of health data for each age group (S103). For example, the reception unit 110 receives reading of the health data stored in the storage device 190 as an input of the health data.

The estimation unit 120 estimates, based on the cost function, the state transition probability based on the transition from the distribution of the health data in the first age group (first distribution) to the distribution of the health data in the second age group (second distribution) (S104). Here, the first age group is in 40s. The second age group is in 50s. That is, the estimation unit 120 estimates the state transition probability based on the transition from the probability density distribution related to the health data of 40s to the probability density distribution related to the health data of 50s. In this case, the cost function is, for example, a function expressing such a cost that the cost required for the transition to the direction in which the value of the blood glucose level increases (the direction in which the blood glucose level deteriorates) decreases as the value of BMI increases.

Then, the output unit 130 outputs the estimated state transition probability (S105). Specifically, the output unit 130 outputs the estimated state transition probability to the storage device 190, and stores the state transition probability in the storage device 190. Alternatively, the output unit 130 may output the estimated state transition probability to a terminal device having a display or the like.

The present operation example is merely an example. That is, the operation of the estimation device 100 of the present disclosure is not limited to this example. The estimation device 100 may not necessarily estimate the state transition probability in consecutive stages. In the present operation example, an example of obtaining the state transition probability from the health data of 40s to the health data of 50s based on the health data in which the age group is classified for every 10s is illustrated. Not limited to this example, for example, the estimation device 100 may obtain the state transition probability from the health data of 40s to the health data of 70s.

As described above, the estimation device 100 of the second example embodiment receives the input of the data set for each stage. The estimation device 100 estimates the state transition probability based on the transition from the first distribution, which is the data distribution in the first stage, to the second distribution, which is the data distribution in the second stage, based on the cost required for the transition to the transition destination determined for each piece of data in the first distribution. Then, the estimation device 100 outputs the estimated state transition probability.

Specifically, the estimation device 100 may receive health data, which is data regarding health at a predetermined time point of each of a plurality of persons for each age group, as a data set for each stage. At this time, the first distribution may be a distribution of health data in a first age group, and the second distribution may be a distribution of health data in a second age group that is an age group after the first age group.

For example, for each piece of health data in the first distribution, a cost relevant to the ease of transition to the transition destination or the like is set, so that the estimation device 100 can estimate the state transition probability based on the transition from the first distribution to the second distribution according to the tendency of the change in data. That is, the estimation device 100 can output the state transition probability in consideration of the tendency of the change in the data in the data transition between the distributions.

In the transition of the health data from the first distribution to the second distribution, the estimation device 100 may estimate the state transition probability based on the transition from the first distribution to the second distribution based on the cost function in which the cost of transition in the direction in which the health data of the first distribution deteriorates is smaller than the cost of transition in the direction in which the health data of the first distribution improves. For example, it is assumed that health data tends to deteriorate easily and hardly improve with aging. The estimation device 100 can estimate the state transition probability in consideration of such a tendency of a change in health data.

It is assumed that the health data includes values of a plurality of health-related items of each of a plurality of persons. Furthermore, it is assumed that the first distribution and the second distribution are multidimensional distributions with each of a plurality of health-related items as an axis. In such a case, in the transition of the health data from the first distribution to the second distribution, the estimation device 100 may estimate the state transition probability based on the transition from the first distribution to the second distribution based on the cost function in which the cost at which the health data of the first distribution transitions differs according to the magnitude of the value relevant to the specific axis. As a result, the estimation device 100 can estimate the state transition probability in consideration of a change in data depending on a value relevant to a specific axis.

### [First Modification]

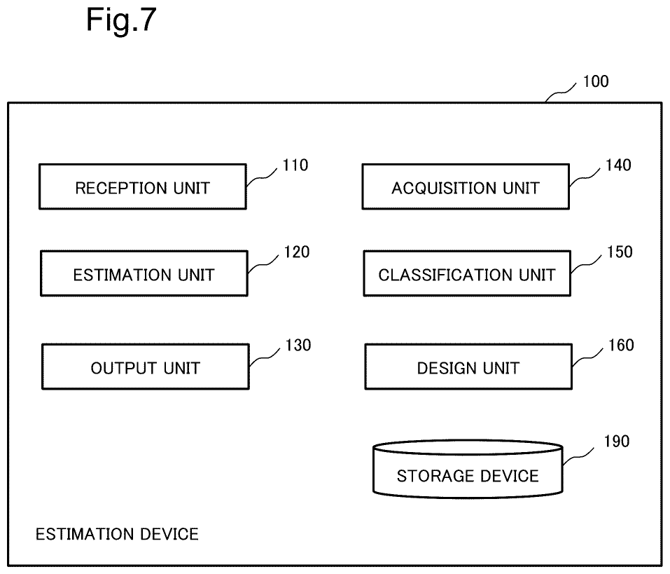

The above-described cost function may be designed in the estimation device 100. FIG. 7 is a block diagram illustrating an exemplary functional configuration of the estimation device 100 of a first modification. In the first modification, the estimation device 100 may further include a design unit 160.

The design unit 160 designs a cost function in advance. For example, the design unit 160 designs a cost function using data accumulated in advance.

Specifically, the design unit 160 designs the function g or the function h in Expressions 7, 8, 10, 11, and 12. At this time, a functional form in which coefficients are determined in advance as parameters is assumed. For example, when parameters are α, β, and γ, the function g(x) can be expressed as the following Expression 14.

$$g ( x ) = α {x}^{2} + β x + γ \qquad [ Math . 14 ]$$

In Expression 14, g(x) is expressed as a quadratic polynomial form. The present invention is not limited to this example, and g(x) may be a linear form, a polynomial form, an exponential form, a logarithmic form, and a functional form of a combination thereof.

For example, in a cost function having the function g(x) of Expression 14, the design unit 160 adjusts the parameters α, β, and γ so as to minimize the total cost calculated by the cost function.

The function g(x) may be modeled by a neural network such as a fully-connected multilayer. In this case, for example, the design unit 160 may obtain the modeled function g(x) by using the total cost as a loss function and performing optimization using machine learning. The function g may be optimized simultaneously with the mapping T. At this time, the machine learning method may be various methods.

It is assumed that health data is accumulated in advance. It is assumed that temporal data, which is a result of the same person being continuously observed for a predetermined period, exists in the accumulated health data. It can be said that the temporal data is data in which correspondence of transitions is clear before and after transition of the age group. Therefore, the design unit 160 may design the cost function using the correspondence data in which correspondence of the transition is clear. For example, the design unit 160 uses a change in data in the distribution of the correspondence data in the age group before transition and the distribution of the correspondence data in the age group after transition to adjust the parameters α, β, and γ so as to match the change.

Also in this case, the design unit 160 may model the function g(x) by a neural network such as a fully-connected multilayer. The design unit 160 may obtain the modeled function g(x) by performing optimization using machine learning with a difference between the correspondence data in the age group before transition and the mapped correspondence data in the age group after transition as a loss function.

Data different from the transition destination can be used as data to which correspondence of the transition is clear. For example, the design unit 160 may use the data of 55 years old in a case where the transition of the age group from 40 years old to 50 years old is learned. For example, there may be data with short-term correspondence of 5 years even if there is no data with long-term correspondence of 10 years or more. In this case, the data of 5 years ahead to be used is not data indicating the transition of 10 years ahead. Therefore, the design unit 160 performs learning by virtually generating a transition 10 years ahead by performing extrapolation twice in accordance with the corresponding time.

Then, the estimation unit 120 estimates the state transition probability based on the transition from the first distribution to the second distribution based on the designed cost function.

The design unit 160 can similarly design the function h.

In this manner, the design unit 160 designs the cost function that calculates the cost determined for each piece of data based on the change in the data between the data distribution in the stage before transition and the data distribution in the stage after transition.

### Third Example Embodiment

Next, an estimation device according to a third example embodiment will be described. In the third example embodiment, an example of predicting the health state of the target person based on the calculated state transition probability will be described. Also in the third example embodiment, an example in which the estimation device estimates the data transition based on the health data will be mainly described, but the target data is not limited to this example. The description of part of the content overlapping with the content of the first example embodiment and the second example embodiment will be omitted.

### <Details of Estimation Device 101>

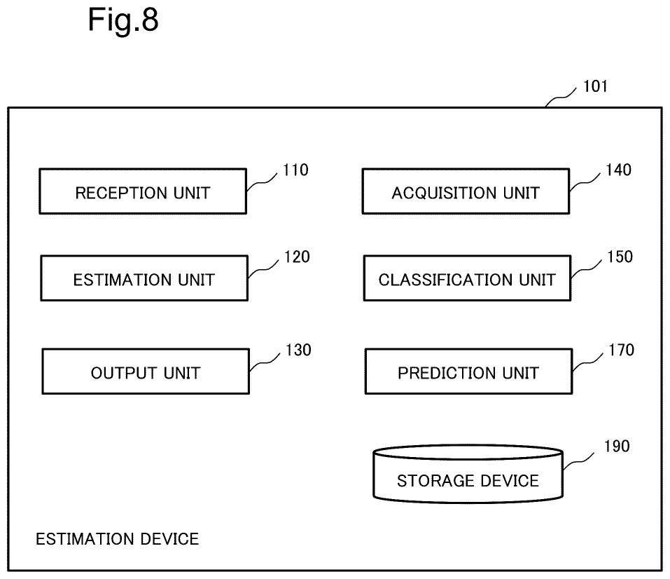

An estimation device 101 is a device in which a further functional unit is added to the estimation device 100. FIG. 8 is a block diagram illustrating an exemplary functional configuration of the estimation device 101. The estimation device 101 includes a reception unit 110, an estimation unit 120, an output unit 130, an acquisition unit 140, and a classification unit 150. The estimation device 101 may include a prediction unit 170. Further, the estimation device 101 may include a storage device 190.

Similarly to the estimation device 100, the estimation device 101 may be a device provided in a terminal device, or may be a device implemented in a server device communicably connected to the terminal device via a wired or wireless network.

The estimation device 101 predicts future health data for the target person using the state transition probability calculated in advance. In the present example embodiment, it is assumed that the first age group is 40s, and the second age group is 50s. Then, it is assumed that a state transition probability from health data in 40s to health data in 50s is calculated.

The acquisition unit 140 acquires health data of the target person. For example, it is assumed that the state transition probability is calculated from the probability density distribution regarding the blood glucose level and BMI. At this time, the acquisition unit 140 acquires health data indicating the blood glucose level and BMI of the target person. The acquired health data regarding the target person is also referred to as target data.

The prediction unit 170 predicts the transition of the health data of the target person. In other words, the prediction unit 170 predicts the value of the future health data of the target person based on the target data. Specifically, the prediction unit 170 predicts the value of the health data in a case where the target person has reached the age relevant to the second age group. At this time, the first age group is an age group relevant to the age of the target person in the target data.

For example, it is assumed that the target person is 41 years old. In this case, the age of the target person is relevant to the first age group. The prediction unit 170 specifies which data group the target data is classified into in the probability density distribution related to the health data in 40s. Then, the prediction unit 170 predicts to which data group the specified data group transitions in the probability density distribution related to the health data in 50s based on the state transition probability.

In this manner, the prediction unit 170 predicts the transition of the target data that is the health data regarding the target person. More specifically, for example, the prediction unit 170 predicts a data group in the second distribution, which is a transition destination based on the state transition probability of the data group in the first distribution relevant to the target data, as the health data in a case where the target person reaches the age of the second age group.

### <Operation Example of Estimation Device 101>

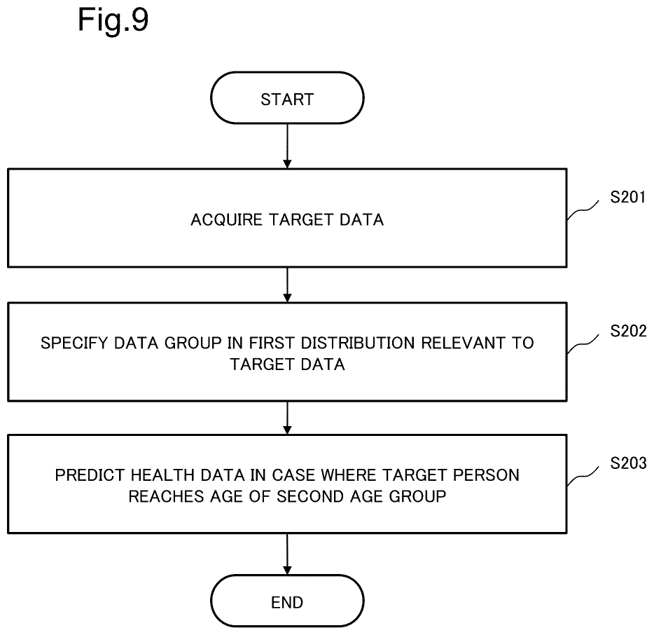

Next, an exemplary operation of the estimation device 101 will be described with reference to FIG. 9.

FIG. 9 is a third flowchart for explaining an exemplary operation of the estimation device 101. Specifically, FIG. 9 is a flowchart illustrating an exemplary operation when the estimation device 101 predicts the health state of the target person.

The acquisition unit 140 acquires target data (S201). For example, the acquisition unit 140 acquires the target data from the terminal device.

The prediction unit 170 specifies a data group in the first distribution relevant to the target data (S202). For example, the prediction unit 170 specifies which data group the target data is classified into in the probability density distribution related to the health data in the first age group.

Then, the prediction unit 170 predicts the health data in a case where the target person reaches the age of the second age group based on the state transition probability (S203). Specifically, the prediction unit 170 predicts a data group in the probability density distribution regarding the health data in the second age group, which is the transition destination based on the state transition probability of the specified data group, as the health data in a case where the target person reaches the age of the second age group.

The present operation example is merely an example. That is, the operation of the estimation device 101 of the present disclosure is not limited to this example.

As described above, the estimation device 101 of the third example embodiment acquires the health data of the target person. At this time, the first age group is an age group relevant to the age of the target person. Then, the estimation device 101 predicts a data group in the second distribution, which is a transition destination based on the state transition probability of the data group in the first distribution relevant to the health data of the target person, as the health data in a case where the target person reaches the age of the second age group. Thus, the estimation device 101 can predict the future health state of the target person.

### Fourth Example Embodiment

Next, an estimation device according to a fourth example embodiment will be described. In the fourth example embodiment, a further example of predicting the health state of the target person based on the calculated state transition probability will be described. Also in the fourth example embodiment, an example in which the estimation device estimates the data transition based on the health data will be mainly described, but the target data is not limited to this example. The description of part of the content overlapping with the content of the first example embodiment, the second example embodiment, and the third example embodiment will be omitted.

### <Details of Estimation Device 102>

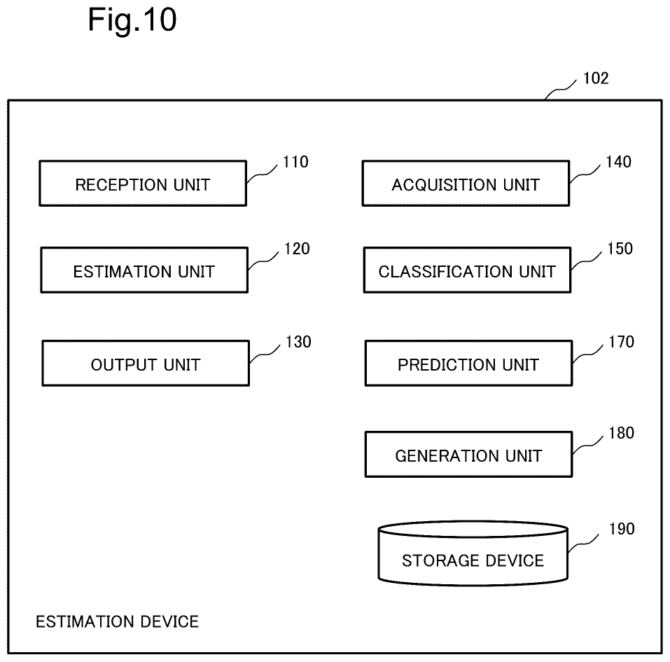

An estimation device 102 is a device in which a further functional unit is added to the estimation device 101. FIG. 10 is a block diagram illustrating an exemplary functional configuration of the estimation device 102. The estimation device 102 includes a reception unit 110, an estimation unit 120, an output unit 130, an acquisition unit 140, a classification unit 150, and a prediction unit 170. The estimation device 101 may include a generation unit 180. Further, the estimation device 102 may include a storage device 190.

Similarly to the estimation device 101, the estimation device 102 may be a device provided in a terminal device, or may be a device implemented in a server device communicably connected to the terminal device via a wired or wireless network.

The estimation device 102 calculates a state transition probability between stages in advance. Then, the estimation device 102 generates a learning model using the calculated state transition probability. The estimation device 102 predicts future health data for the target person using the generated learning model. In the present example embodiment, a stage of generating the learning model is referred to as a generation phase. A stage of performing prediction is referred to as a prediction phase.

### (Generation Phase)

It is assumed that a data set for each stage is stored in the storage device 190 in advance. For example, it is assumed that a probability density distribution related to health data for each age group is stored in the storage device 190.

The reception unit 110 receives an input of a probability density distribution related to health data for each age group. In the present example embodiment, the probability density distribution is a distribution related to health data for every 10s. It is assumed that there are eight types of probability density distributions from 10s to 80s.

The estimation unit 120 estimates a state transition probability related to each stage. For example, the estimation unit 120 estimates the state transition probability based on the transition from the probability density distribution related to the health data of 10s to the probability density distribution related to the health data of 20s. The estimation unit 120 estimates the state transition probability based on the transition from the probability density distribution related to the health data of 20s to the probability density distribution related to the health data of 30s. Similarly, the estimation unit 120 estimates the state transition probability based on the transition of the data distribution for each stage based on the probability density distribution regarding the health data between the stages. At this time, the estimation unit 120 may also estimate a state transition probability between stages that are not adjacent. For example, the estimation unit 120 may estimate the state transition probability based on the transition from the probability density distribution related to the health data of 20s to the probability density distribution related to the health data of 50s.

The output unit 130 outputs the state transition probability related to the data transition between the stages. At this time, the output unit 130 may store the state transition probability and a set of stages relevant to the state transition probability in the storage device 190 in association with each other.

The generation unit 180 generates a machine learning model. Specifically, the generation unit 180 generates a prediction model that outputs data in another stage, which is a transition destination of data in one stage, based on the output state transition probability. The prediction model is relevant to a machine learning model that learns a relationship between a data distribution in one stage and a data distribution in another stage. Alternatively, the generation unit 180 may generate a prediction model that outputs a data group of data in another stage, which is a transition destination of a data group of a data distribution in one stage, based on the output state transition probability. The generated prediction model is a machine learning model that receives age and health data as inputs, and outputs health data in which the input health data transitions after a predetermined period or a data group thereof.

In this manner, the generation unit 180 generates the machine learning model in which the relationship between the data in the stage before transition and the data in the stage after transition is learned based on the state transition probability. More specifically, the generation unit 180 generates a machine learning model in which the relationship between the health data in the age group before transition and the health data in the age group after transition is learned based on the state transition probability.

### (Prediction Phase)

The acquisition unit 140 acquires target data that is health data of a target person.

The prediction unit 170 estimates the future health state of the target person using the machine learning model. Specifically, the prediction unit 170 predicts the transition of the target data, which is the health data regarding the target person. For example, the prediction unit 170 inputs the target data and the age of the target person to the machine learning model. The health data in the age group equal to or higher than the age of the target person is output by the machine learning model. That is, the health data in a case where the target person reaches the age after the lapse of the predetermined period is output. The prediction unit 170 predicts the health data as future health data of the target person.

For example, it is assumed that the target person is 51 years old. Then, it is assumed that health data in a case where the target person is in 70s is predicted. In this case, the prediction unit 170 inputs, for example, information indicating that the age is 51 years old and the target data to the machine learning model. At this time, the machine learning model outputs the health data in the age group of 70s, which is the transition destination of the input health data. The prediction unit 170 outputs the output health data as health data in a case where the target person is in 70s.

The machine learning model may be a model that outputs health data after a lapse of a specific period with respect to the target data. For example, the machine learning model may output health data in a case where the target person reaches an age group after 20 years. The machine learning model may be a model that outputs a transition of health data until after a lapse of a specific period. For example, the transition of the health data until the target person reaches the age group after 20 years may be output.

### <Operation Example of Estimation Device 102>

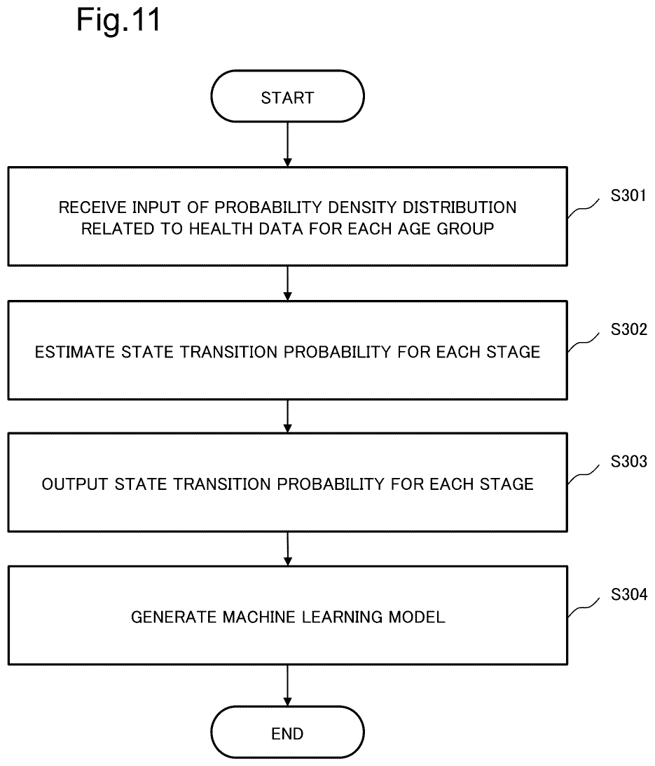

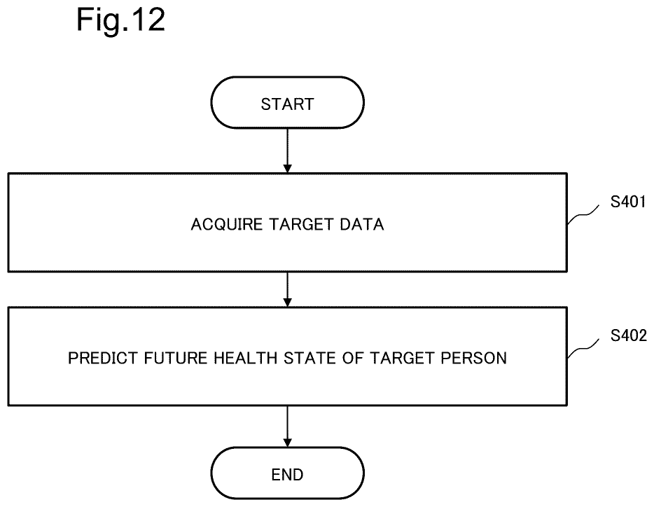

Next, an example of the operation of the estimation device 102 will be described with reference to FIGS. 11 and 12.

FIG. 11 is a fourth flowchart for explaining an exemplary operation of the estimation device 102. Specifically, FIG. 11 is a flowchart for explaining an example of the operation of the estimation device 102 in the generation phase. In the operation example of FIG. 11, it is assumed that a probability density distribution related to health data for each age group is stored in the storage device 190 in advance.

The reception unit 110 receives an input of a probability density distribution related to health data for each age group (S301). For example, the reception unit 110 receives an input of a probability density distribution related to health data for every 10s.

The estimation unit 120 estimates the state transition probability for each of the stages (S302). Specifically, the estimation unit 120 estimates the state transition probability based on the transition of the data distribution for each of the stages based on the probability density distribution related to the health data for each of the stages.

The output unit 130 outputs the state transition probability for each stage (S303). Then, the generation unit 180 generates a machine learning model based on the output state transition probability (S304). Specifically, the generation unit 180 generates the machine learning model in which the relationship between the data in the stage before transition and the data in the stage after transition is learned based on the state transition probability.

FIG. 12 is a fifth flowchart for explaining an exemplary operation of the estimation device 102. Specifically, FIG. 12 is a flowchart for explaining an example of the operation of the estimation device 102 in the prediction phase.

The acquisition unit 140 acquires target data (S401). For example, the acquisition unit 140 acquires the target data from the terminal device.

The prediction unit 170 predicts the future health state of the target person using the machine learning model (S402). Specifically, the prediction unit 170 inputs information indicating the age of the target person and the target data to the machine learning model. Then, the health data is output by the machine learning model. The prediction unit 170 outputs the output health data as health data in a case where the target person reaches an age after a predetermined period has elapsed.

The present operation example is merely an example. That is, the operation of the estimation device 102 of the present disclosure is not limited to this example.

### [Second Modification]

In the present disclosure, the example in which the estimation device estimates the data transition based on the health data has been mainly described. That is, an example in which the estimation device is used in the healthcare or medical field has been mainly described. However, the example to which the estimation device is applied is not limited thereto. For example, the estimation device may also be applied to a case of estimating state transitions of various machines.

For example, in a case where the measurement data measured for the operating state of the machine is acquired, the estimation device may receive, as the data set for each stage, the measurement data of each state based on the secular change from the state in which the machine normally operates to the state in which the machine fails. The estimation device may estimate the state transition probability based on the distribution of the measurement data between the states. Then, the estimation device may output the state transition probability related to the transition between the states.

### <Exemplary Hardware Configuration of Estimation Device>

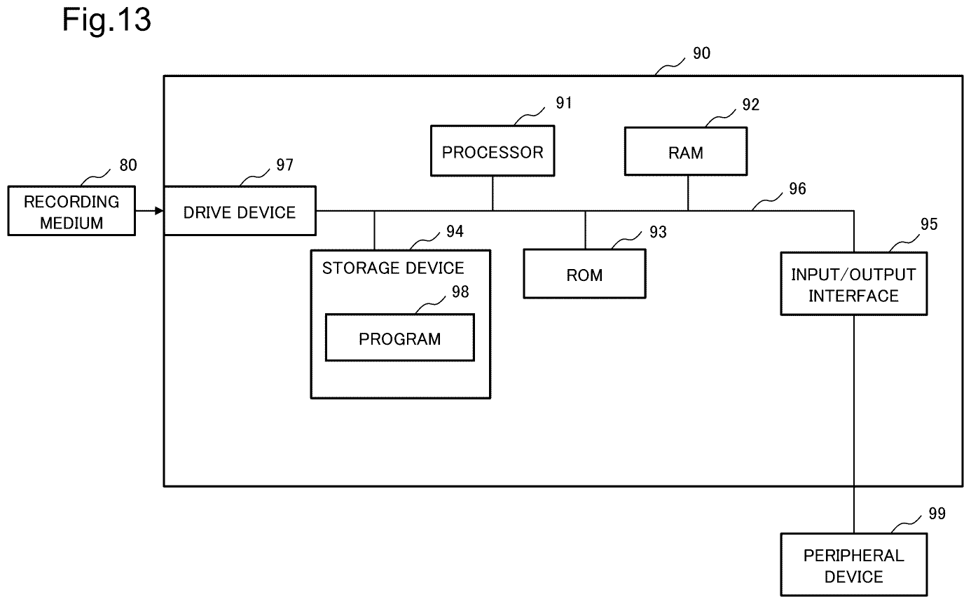

Hardware constituting the estimation devices of the first, second, third, and fourth example embodiments will be described. FIG. 13 is a block diagram illustrating an example of a hardware configuration of a computer device constituting the estimation device according to each example embodiment. In a computer device 90, the estimation device and the estimation method described in each example embodiment and each modification are achieved. For example, the estimation device and the like described in each example embodiment and each modification may have the hardware configuration illustrated in FIG. 13.

As illustrated in FIG. 13, the computer device 90 includes a processor 91, a random access memory (RAM) 92, a read only memory (ROM) 93, a storage device 94, an input/output interface 95, a bus 96, and a drive device 97. The estimation device and the like may be achieved by a plurality of electric circuits.

The storage device 94 stores a program (computer program) 98. The processor 91 executes the program 98 of the present estimation device using the RAM 92. Specifically, for example, the program 98 includes a program that causes a computer to execute the processing illustrated in FIG. 2, FIG. 6, FIG. 9, FIG. 11, and FIG. 12. When the processor 91 executes the program 98, the function of each configuration of the present estimation device is implemented. The program 98 may be stored in the ROM 93. The program 98 may be recorded in a recording medium 80 and read using a drive device 97, or may be transmitted from an external device (not illustrated) to the computer device 90 via a network (not illustrated).

The input/output interface 95 exchanges data with a peripheral device (keyboard, mouse, display device, etc.) 99. The input/output interface 95 functions as a means for acquiring or outputting data. The bus 96 connects the components.

There are various modifications of the method of achieving the estimation device. For example, each configuration included in the estimation device can be achieved as a dedicated device. The estimation device can be achieved based on a combination of a plurality of devices.

A processing method of causing a recording medium to record a program for achieving each configuration in the functions of each example embodiment, reading the program recorded in the recording medium as a code, and a computer executing the program are also included in the scope of each example embodiment. That is, a computer-readable recording medium is included in the scope of each example embodiment. A recording medium recording the above-described program and the program itself are also included in each example embodiment.

The recording medium is, for example, a floppy (registered trademark) disk, a hard disk, an optical disk, a magneto-optical disk, a compact disc (CD)-ROM, a magnetic tape, a nonvolatile memory card, or a ROM, but is not limited to this example. The program recorded in the recording medium is not limited to a program that executes processing by itself, and programs that operate on an operating system (OS) to execute processing in cooperation with other software and functions of an extension board are also included in the scope of each example embodiment.

Some or all of the above example embodiments may be described as the following Supplementary Notes, but are not limited to the following.

In the case of estimating the state transition, the ease of transition to the transition destination may vary depending on the type of data to be handled, the value of the data, or the like. For example, there is a possibility that the test value related to the health state by the medical examination or the like has a stronger tendency to deteriorate with aging than the tendency to improve. As such, the data may have a particular trend of change.

An object of the present disclosure is to provide an estimation device and the like capable of outputting a state transition probability in consideration of a tendency of a change in data in data transition between distributions.

According to the present disclosure, in data transition between distributions, it is possible to output a state transition probability in consideration of a tendency of data change.

### <Supplementary Notes>

### [Supplementary Note 1]

An estimation device including:
a reception unit that receives an input of a data set for each stage;
an estimation unit that estimates a state transition probability based on a transition from a first distribution that is a data distribution in a first stage to a second distribution that is a data distribution in a second stage based on a cost determined for each piece of data in the first distribution and required for a transition to a transition destination; and
an output unit that outputs an estimated state transition probability.

### [Supplementary Note 2]

The estimation device according to Supplementary Note 1, in which
the reception unit receives health data, which is data regarding health at a predetermined time point of each of a plurality of persons for each age group, as a data set for each stage,
the first distribution is a distribution of health data in a first age group, and
the second distribution is a distribution of health data in a second age group that is an age group after the first age group.

### [Supplementary Note 3]

The estimation device according to Supplementary Note 2, in which
in a transition of health data from the first distribution to the second distribution, the estimation unit estimates a state transition probability based on a transition from the first distribution to the second distribution based on a cost function in which a cost of transition in a direction in which health data of the first distribution deteriorates is smaller than a cost of transition in a direction in which health data of the first distribution improves.

### [Supplementary Note 4]

The estimation device according to Supplementary Note 2, in which
the health data includes values of a plurality of health-related items for each of a plurality of persons,
the first distribution and the second distribution are multidimensional distributions having a plurality of health-related items as axes, and
in a transition of health data from the first distribution to the second distribution, the estimation unit estimates a state transition probability based on a transition from the first distribution to the second distribution, based on a cost function in which a cost at which health data of the first distribution transitions differs according to a magnitude of a value relevant to a specific axis.

### [Supplementary Note 5]

The estimation device according to Supplementary Note 1, including a design unit that designs a cost function that calculates a cost determined for each piece of data based on a change in data between a data distribution in a stage before transition and a data distribution in a stage after transition, in which
the estimation unit estimates a state transition probability based on a transition from the first distribution to the second distribution based on the generated cost function.

### [Supplementary Note 6]

The estimation device according to Supplementary Note 5, in which
the design unit designs a cost function including a function in which a parameter is adjusted according to a change between correspondence data in a stage before transition and correspondence data in a stage after transition based on correspondence data in which correspondence of transition is known.

### [Supplementary Note 7]

The estimation device according to Supplementary Note 1, in which
the estimation unit estimates a state transition probability by using an optimal transport algorithm that optimizes a cost for transport from the first distribution to the second distribution and calculates a set of data before transition and data of a transport destination.

### [Supplementary Note 8]

The estimation device according to Supplementary Note 2, further including:
a generation unit that generates a machine learning model; and
a prediction unit that predicts a transition of target data that is health data on a target person, in which
the estimation unit estimates a state transition probability based on a transition of a distribution of health data for each age group,
the output unit outputs a state transition probability for each age group,
the generation unit generates the machine learning model in which a relationship between health data in an age group before transition and health data in an age group after transition is learned based on a state transition probability, and
the prediction unit predicts a transition of target data using the machine learning model.

### [Supplementary Note 9]

An estimation method including:
receiving an input of a data set for each stage;
estimating a state transition probability based on a transition from a first distribution that is a data distribution in a first stage to a second distribution that is a data distribution in a second stage based on a cost determined for each piece of data in the first distribution and required for a transition to a transition destination; and
outputting an estimated state transition probability.

### [Supplementary Note 10]

A non-transitory recording medium recording a program for causing a computer to execute:
a process of receiving an input of a data set for each stage;
a process of estimating a state transition probability based on a transition from a first distribution that is a data distribution in a first stage to a second distribution that is a data distribution in a second stage based on a cost determined for each piece of data in the first distribution and required for a transition to a transition destination; and
a process of outputting an estimated state transition probability.

Some or all of the configurations described in Supplementary Notes 2 to 8 dependent on the above-described Supplementary Note 1 can also be dependent on Supplementary Notes 9 and 10 by the same dependency relationship as in Supplementary Notes 2 to 8. Some or all of the configurations described as a Supplementary Note can be similarly dependent on various recording means or systems for recording various hardware, software, and software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. An estimation device comprising:
at least one memory storing instructions; and
at least one processor configured to access the at least one memory and execute the instructions to:
receive an input of a data set for each stage;
estimate a state transition probability based on a transition from a first distribution that is a data distribution in a first stage to a second distribution that is a data distribution in a second stage based on a cost determined for each piece of data in the first distribution and required for a transition to a transition destination; and
output an estimated state transition probability.

2. The estimation device according to claim 1, wherein
the at least one processor is further configured to execute the instructions to:
receive health data, which is data regarding health at a predetermined time point of each of a plurality of persons for each age group, as a data set for each stage, wherein
the first distribution is a distribution of health data in a first age group, and
the second distribution is a distribution of health data in a second age group that is an age group after the first age group.

3. The estimation device according to claim 2, wherein
the at least one processor is further configured to execute the instructions to:
estimate, in a transition of health data from the first distribution to the second distribution, a state transition probability based on a transition from the first distribution to the second distribution based on a cost function in which a cost of transition in a direction in which health data of the first distribution deteriorates is smaller than a cost of transition in a direction in which health data of the first distribution improves.

4. The estimation device according to claim 2, wherein
the at least one processor is further configured to execute the instructions to:
estimate, in a transition of health data from the first distribution to the second distribution, a state transition probability based on a transition from the first distribution to the second distribution, based on a cost function in which a cost at which health data of the first distribution transitions differs according to a magnitude of a value relevant to a specific axis, wherein
the health data includes values of a plurality of health-related items for each of a plurality of persons, and
the first distribution and the second distribution are multidimensional distributions having a plurality of health-related items as axes.

5. The estimation device according to claim 1,
the at least one processor is further configured to execute the instructions to:
design a cost function that calculates a cost determined for each piece of data based on a change in data between a data distribution in a stage before transition and a data distribution in a stage after transition; and
estimate a state transition probability based on a transition from the first distribution to the second distribution based on the generated cost function.

6. The estimation device according to claim 5, wherein
the at least one processor is further configured to execute the instructions to:
design a cost function including a function in which a parameter is adjusted according to a change between correspondence data in a stage before transition and correspondence data in a stage after transition based on correspondence data in which correspondence of transition is known.

7. The estimation device according to claim 1, wherein
the at least one processor is further configured to execute the instructions to:
estimate a state transition probability by using an optimal transport algorithm that optimizes a cost for transport from the first distribution to the second distribution and calculates a set of data before transition and data of a transport destination.

8. The estimation device according to claim 2, wherein
the at least one processor is further configured to execute the instructions to:
generate a machine learning model;
predict a transition of target data that is health data on a target person;
estimate a state transition probability based on a transition of a distribution of health data for each age group;
output a state transition probability for each age group;
generate the machine learning model in which a relationship between health data in an age group before transition and health data in an age group after transition is learned based on a state transition probability; and
predict a transition of target data using the machine learning model.

9. An estimation method comprising:
receiving an input of a data set for each stage;
estimating a state transition probability based on a transition from a first distribution that is a data distribution in a first stage to a second distribution that is a data distribution in a second stage based on a cost determined for each piece of data in the first distribution and required for a transition to a transition destination; and
outputting an estimated state transition probability.

10. A non-transitory recording medium recording a program for causing a computer to execute:
a process of receiving an input of a data set for each stage;
a process of estimating a state transition probability based on a transition from a first distribution that is a data distribution in a first stage to a second distribution that is a data distribution in a second stage based on a cost determined for each piece of data in the first distribution and required for a transition to a transition destination; and
a process of outputting an estimated state transition probability.
